(19) United States
(12) Patent Application Publication
(10) Pub. No.: US 2009/0099461 A1
(43) Pub. Date: Apr. 16, 2009

(54) SYSTEM AND METHOD FOR A NON-SUPINE EXTREMITY BLOOD PRESSURE RATIO EXAMINATION

(75) Inventors: David C. Jones, Evergreen, CO (US); Kenneth E. Jarrell, Littleton, CO (US)

Correspondence Address: MARSH, FISCHMANN & BREYFOGLE LLP, 8055 East Tufts Avenue, Suite 450, Denver, CO, 80237, US

(73) Assignee: Summit Doppler Systems, Inc., Golden, CO (US)

(21) Appl. No.: 12/179,374

(22) Filed: Jul. 24, 2008

Related U.S. Application Data
(60) Provisional application No. 60/980,085, filed on Oct. 15, 2007.

(51) Int. Cl. A61B 5/02; A61B 5/022

(52) U.S. Cl. 600/483; 600/492

(57) ABSTRACT

The present invention provides methods that facilitate the determination of a hydrostatic correction factor usable in an EBPR examination of a patient in a non-supine position. In one embodiment, a method for facilitating the determination of a hydrostatic correction factor for an extremity blood pressure ratio examination of a patient in a non-supine position includes establishing the vertical distance between first and second blood pressure measuring devices positionable on first and second extremities of the patient based on an empirically derived formula and determining a hydrostatic correction factor for the extremity blood pressure ratio examination based on the vertical distance. In this regard, the formula for the vertical distance may be based on a percentage of the height of the patient.

### RELATED APPLICATION INFORMATION

This application claims priority from U.S. Provisional Application Ser. No. 60/980,085, entitled “SYSTEM AND METHOD FOR A NON-SUPINE EXTREMITY BLOOD PRESSURE RATIO EXAMINATION” filed on Oct. 15, 2007, which is incorporated by reference herein in its entirety.

### FIELD OF THE INVENTION

The present invention relates generally to blood pressure examination of a patient, and more particularly to extremity blood pressure ratio examination of a patient in a non-supine position.

### BACKGROUND

Atherosclerosis of the lower extremities, also known as peripheral arterial disease (P.A.D), is a highly prevalent condition affecting about 5% of adults over 50 years of age in the United States. A typical symptom of P.A.D. is pain in the legs during exertion that is relieved with rest. One method of diagnosing P.A.D. is to compare the blood pressure at two patient extremities (e.g., leg and arm blood pressures). This method is generally referred to herein as an extremity blood pressure ratio (EBPR) examination. One such common EBPR procedure for the diagnosis of P.A.D. is the ankle brachial index (ABI) examination. The ABI exam compares blood pressure at the arm with blood pressure at the ankle. When the ankle and arm systolic pressures are obtained, the ratio of ankle pressure to arm pressure is normally greater than 1.0. An ankle/brachial ratio (ABI) that is less than 0.9 is considered abnormal.

Normally, the ABI exam is performed while the patient is in a supine position because the reference for indirect blood pressure measurement is typically the hydrostatic level of the right atrium of a patient's heart (although the left ventricle is sometimes referenced). This is why blood pressure measurements are typically taken at a patient's upper arm, which is approximately the level of their right atrium (or left ventricle). If the limb subject to measurement is at a level below the right atrium (or left ventricle), the pressure will be higher due to the force exerted by hydrostatic pressure. Therefore when an ABI exam is performed on the patient in a supine position, the patient's ankle and arm are approximately level with the right atrium (or left ventricle).

### SUMMARY OF THE INVENTION

One object of the present invention is to facilitate performance of EBPR examinations on patients in non-supine positions (e.g., seated, standing, resting in an elevated bed). It may be desirable in certain circumstances to be able to perform the EBPR examination in a non-supine position if, for example, the patient is confined to a wheelchair, is morbidly obese, has degenerative back problems, or the like. Additionally, the ability to perform the EBPR examination in a non-supine position may lead to faster, easier methods for performing the exam in a physician's office.

The present invention recognizes that in order to perform an EBPR exam on a patient in a non-supine position, a correction factor based on the specific gravities of blood and mercury may be applied to correct for an increased hydrostatic pressure in one or more extremities of the patient. The correction factor for the non-supine EBPR exam depends on the vertical distance between blood pressure cuffs positioned on the patient's extremities. Specifically, the increase in the blood pressure of an extremity due to hydrostatic pressure is equal to the vertical distance between the blood pressure measuring device positioned on the extremity and the patient's heart multiplied by the ratio of the specific gravities of blood and mercury. Once this increased pressure is known, it can be subtracted from the measured extremity blood pressure to provide a corrected extremity blood pressure. Subsequently, the EBPR for a patient may be calculated using conventional methods.

Accordingly, a first aspect of the present invention generally relates to an apparatus that facilitates the determination of a hydrostatic correction factor usable in an EBPR examination of a patient in a non-supine position. A first blood pressure measuring device may be positionable on a first extremity of the patient, and a second blood pressure measuring device may be positionable on a second extremity of the patient, wherein one of the blood pressure measuring devices is located above the other one. The apparatus includes a locating mechanism that fixes a position of at least one of the first and second blood pressure measuring devices relative to its respective patient extremity. The apparatus also includes an instrumentality that provides information about a vertical distance between the first and second blood pressure measuring devices. The vertical distance is usable in determining the hydrostatic correction factor.

A second aspect of the present invention generally relates to an apparatus that facilitates the determination of a hydrostatic correction factor usable in an EBPR examination of a patient in a non-supine position. A first blood pressure measuring device may be positionable on a first extremity of the patient, and a second blood pressure measuring device may be positionable on a second extremity of the patient, wherein one of the blood pressure measuring devices is located above the other one. The apparatus includes an instrumentality coupled to at least one of the first and second blood pressure measuring devices which provides information about a vertical distance between the first and second blood pressure measuring devices. The vertical distance is usable in determining the hydrostatic correction factor.

A third aspect of the present invention generally relates to a method for facilitating the determination of a hydrostatic correction factor for an EBPR examination of a patient in a non-supine position. A first blood pressure measuring device may be positionable on a first extremity of the patient, and a second blood pressure measuring device may be positionable on a second extremity of the patient, wherein one of the blood pressure measuring devices is located above the other one. The method includes positioning a locating mechanism relative to the patient, wherein the locating mechanism fixes a portion of at least one of the first and second blood pressure measuring devices positioned relative to its respective patient extremity. The method also includes measuring the vertical distance between the first and second blood pressure measuring devices, and determining a hydrostatic correction factor for the EBPR examination.

A fourth aspect of the present invention generally relates to a method for facilitating the determination of a hydrostatic correction factor for an EBPR examination of a patient in a non-supine position. A first blood pressure measuring device may be positionable on a first extremity of the patient, and a second blood pressure measuring device may be positionable on a second extremity of the patient, wherein one of the blood pressure measuring devices is located above the other one. The method includes establishing the vertical distance between the first and second blood pressure measuring devices based on an empirically derived formula. The method further includes determining a hydrostatic correction factor for the EBPR examination based on the vertical distance. The empirically derived formula for the vertical distance may, for example, be based on a percentage of the height of the patient.

A fifth aspect of the present invention generally relates to an apparatus that facilitates the determination of a hydrostatic correction factor usable in an EBPR examination of a patient in a non-supine position. A first blood pressure measuring device may be positionable on a first extremity of the patient, and a second blood pressure measuring device may be positionable on a second extremity of the patient, wherein one of the blood pressure measuring devices is located above the other one. The apparatus includes a computational device, and an input element coupled to the computational device. Information relating to a vertical distance between the first and second blood pressure measuring devices may be receivable by the input element. The computational device is also operable to calculate at least one of a hydrostatic correction factor, corrected extremity blood pressure, and EBPR using the information relating to the vertical distance. Furthermore, the computational device may be coupled to an output element that is operable to output information relating to the EBPR examination of the patient.

A sixth aspect of the present invention generally relates to a method for facilitating the determination of a hydrostatic correction factor for an EBPR examination of a patient in a non-supine position. A first blood pressure measuring device may be positionable on a first extremity of the patient, and a second blood pressure measuring device may be positionable on a second extremity of the patient, wherein one of the blood pressure measuring devices is located above the other one. The method includes receiving information relating to a vertical distance between the first and second blood pressure measuring devices, and determining a hydrostatic correction factor for the EBPR examination using the information relating to the vertical distance.

Various refinements exist of the features noted in relation to the various aspects of the present invention. Further features may also be incorporated in the various aspects of the present invention. These refinements and additional features may exist individually or in any combination, and various features of the various aspects may be combined. For example, the locating mechanism may include a rod, and an arm member coupled to the rod and one of the blood pressure measuring devices. The arm member may also be movable along a portion of the rod. Furthermore the instrumentality may include markings on the rod that correspond to a distance from a reference point which enables an operator to determine the vertical distance between the blood pressure measuring devices. Additionally, the instrumentality may include a mechanism for automatically measuring the vertical distance. For example, the instrumentality may include an optical sensing device, an ultrasonic sensing device, an electromechanical sensing device, or the like. Additionally, a processor may be coupled to the instrumentality that is operable to calculate the hydrostatic correction factor based on the information received from the instrumentality. The processor may also be operable to receive blood pressure information and to calculate at least one of a vertical distance, a hydrostatic correction factor, an EBPR, and a corrected extremity pressure of the patient. Furthermore, the processor may be coupled to an output device that is operable to output information relating to an EBPR examination.

In the various aspects of the present invention, one of the blood pressure measuring devices is located above the other blood pressure measuring device. In this regard, one of the blood pressure measuring devices may, for example, be on the patient's arm (e.g., their upper arm) and the other blood pressure measuring device may, for example, be on the patient's leg (e.g., their ankle) on the same or opposite side of the patient, although it may also be possible for the blood pressure measuring devices to be on opposing arms or opposing legs (e.g., on the thigh of one leg and the ankle of the other leg or the upper arm on one side of the patient and the wrist on the other side of the patient). Further, there may be more than two blood pressure measuring devices (e.g., one on the patient's arm, one on the patients' thigh and one on the patient's ankle).

In the various aspects of the invention, the blood pressure measuring devices may take various forms including, for example, devices that include blood pressure cuffs, devices that do not necessarily use cuffs such as devices that employ vascular unloading, direct pressure measurement (e.g. catheter) and pulse wave velocity techniques to measure patient blood pressure, and devices that measure patient blood pressure directly.

These and other aspects and advantages of the present invention will be apparent upon review of the following Detailed Description when taken in conjunction with the accompanying figures.

### BRIEF DESCRIPTION OF THE DRAWINGS

For a more complete understanding of the present invention and further advantages thereof, reference is now made to the following Detailed Description, taken in conjunction with the drawings, in which:

FIGS. 1A-1C illustrate an embodiment of an apparatus for facilitating a seated ABI or other non-supine EBPR examination;

FIGS. 2A-2B illustrate an embodiment of an apparatus for facilitating a seated ABI or other non-supine EBPR examination that includes an optical sensing device;

FIGS. 2C-2D illustrate another embodiment of an apparatus for facilitating a seated ABI or other non-supine EBPR examination that includes an optical sensing device;

FIG. 3 illustrates an embodiment of an apparatus for facilitating a seated ABI or other non-supine EBPR examination that includes an ultrasonic sensing device;

FIG. 4 illustrates an embodiment of an apparatus for facilitating a seated ABI or other non-supine EBPR examination that includes an electromechanical sensing device;

FIG. 5 illustrates an embodiment of an apparatus for facilitating a seated ABI or other non-supine EBPR examination that includes an examination chair;

FIG. 6 is a block diagram of a system for facilitating a seated ABI or other non-supine EBPR examination;

FIG. 7 is a chart that illustrates empirical data of the ratio of the vertical distance between two blood pressure cuffs positioned on a seated patient to their height;

FIG. 8 illustrates the steps of one embodiment of a method for facilitating the determination of a hydrostatic correction factor for a seated ABI or other non-supine EBPR examination of a patient;

FIG. 9 illustrates the steps of another embodiment of a method for facilitating the determination of a hydrostatic correction factor for a seated ABI or other non-supine EBPR examination of a patient; and

FIG. 10 illustrates the steps of a further embodiment of a method for facilitating the determination of a hydrostatic correction factor for a seated ABI or other non-supine EBPR examination of a patient.

### DETAILED DESCRIPTION OF THE DRAWINGS

While the invention is susceptible to various modifications and alternative forms, specific embodiments thereof have been shown by way of example in the drawings and are herein described in detail. It should be understood, however, that it is not intended to limit the invention to the particular form disclosed, but rather, the invention is to cover all modifications, equivalents, and alternatives falling within the scope and spirit of the invention as defined by the claims.

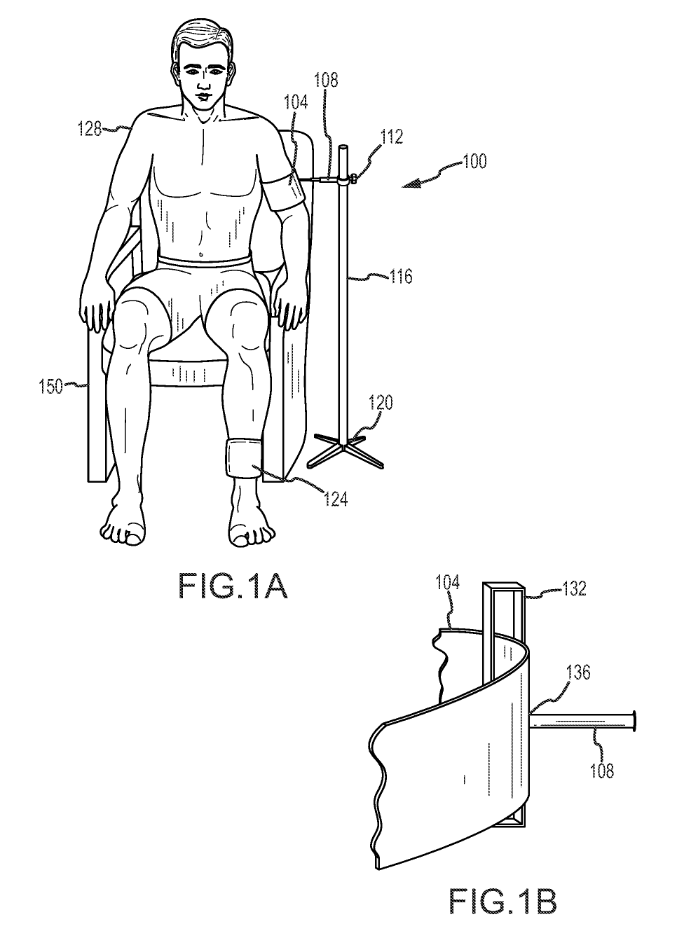

FIGS. 1A-1C illustrate one embodiment of a seated ABI apparatus (SAA) 100. In this embodiment, a patient 128 may sit in a chair 150 so that the ABI exam may be administered by an operator (e.g., a health care worker). The operator may secure a blood pressure cuff 124 to the ankle of the patient 128. Similarly, the operator may secure another blood pressure cuff 104 to the arm of the patient 128. The arm of patient 128 may be desirably positioned and the arm blood pressure cuff 104 desirably secured thereon such that the arm blood pressure cuff 104 is approximately level with the patient's heart. Disposed next to the patient is a stand rod 116 which is held upright by a stand base 120. Furthermore, a slide ring 112 may be slidably coupled to the stand rod 116. The arm blood pressure cuff 104 may be coupled to the slide ring 112 through a rod 108 and a cuff holding frame 132.

As shown in FIG. 1B, the operator may secure the blood pressure cuff 104 to the SAA 100 by threading it through an opening in the cuff holding frame 132. Furthermore, the cuff holding frame 132 may be adjustable relative to the rod 108 by the pivot 136 to permit the SAA 100 to fit patients with various body types and builds.

FIG. 1C illustrates the operation of the slide ring 112 on the stand rod 116. The slide ring 112 may be temporarily fixed at a position on the stand rod 116 by tightening a tightening knob 144. In this regard, the slide ring 112 may move freely up and down the stand rod 116 when the operator loosens the tightening knob 144. As shown in FIG. 1C, the stand rod 116 may include graticules 140 which allow the operator to read the height of slide ring 112 which corresponds to the height of the blood pressure cuff 104 relative to a reference point (e.g., the bottom of the stand base 120). Then, once the height of the blood pressure cuff 124 positioned on the ankle of the patient is known, the vertical distance between the blood pressure cuffs 104, 124 may be calculated. The height of the blood pressure cuff 124 positioned on the ankle may be measured in various manners (e.g., using another stand rod and stand base, using a tape measure, or the like). Alternatively or additionally, the height of blood pressure cuff 124 may be estimated to be the same for all patients (e.g., 8 inches from the ground).

The operator may calculate the hydrostatic correction factor (HCF) in any number of ways. For example, the operator may use the formula described above to calculate the HCF by hand, by using a chart, or by using a conventional calculator. Alternatively or additionally, the vertical distance between the blood pressure cuffs 104, 124 may be provided to a computational device that is operable to calculate the HCF and/or the ABI of the patient. To calculate the ABI, the blood pressure measurements from each of the blood pressure cuffs 104, 124 may be provided in addition to the vertical distance.

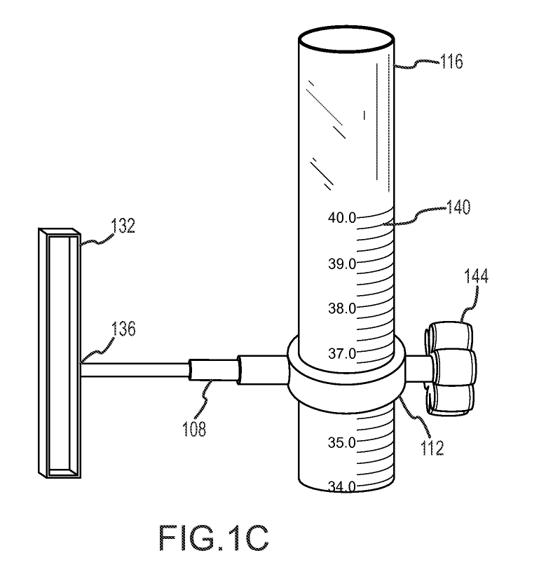

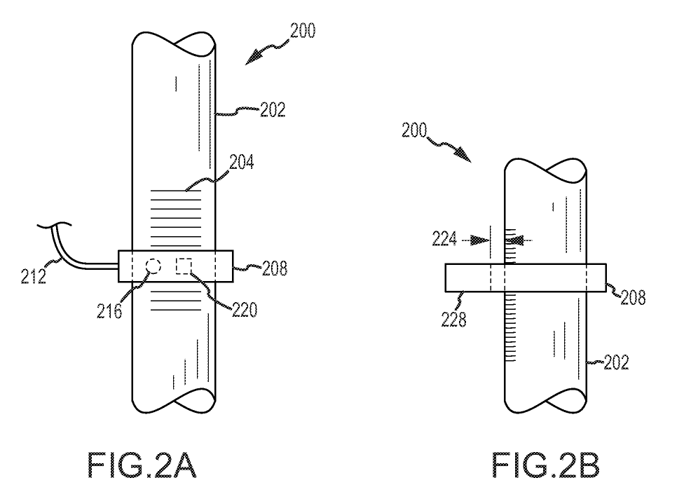

FIGS. 2A-2B illustrate an embodiment of an SAA 200 in which the vertical distance between the blood pressure cuffs may be measured using an optical sensing mechanism. The SAA 200 may be used with blood pressure cuffs or other suitable blood pressure measuring devices positionable on extremities of a patient in a manner similar to that depicted, for example, in FIG. 1A. Similar to the previously described embodiment of FIGS. 1A-1C, a slide ring 208 is slidably coupled to a stand rod 202. The slide ring 208 may be temporarily fixed to the stand rod 202 by a tightening knob (e.g., the tightening knob 144 of FIG. 1C). The slide ring 208 may include an optical emitter 216 and an optical detector 220 operable to sense the position of the slide ring 208 relative to the stand rod 202. The stand rod 202 may include markings 204 which enable the optical emitter-detector pair 216, 220 to differentiate between various heights along the stand rod 202. As shown in FIG. 2B, the optical emitter-detector pair 216, 220 may be positioned on an emitter-detector mounting area 228 of the slide ring 208. The emitter-detector mounting area 228 will preferably be positioned such that there is an optical gap 224 between the emitter-detector mounting area 228 and the stand rod 202. This permits the optical signals to be transmitted by the optical emitter 216, and the reflected signals to be received by the optical detector 220. The optical emitter-detector pair 216, 220 may communicate the position of the slide ring 208 on the stand rod 202 to a computational device (not shown) via cable 212. The computational device may be operable to output the height of a blood pressure cuff or the vertical distance between two blood pressure cuffs. Furthermore, the computational device may be operable to determine the HCF and/or the ABI for the patient.

FIGS. 2C-2D illustrate an embodiment of an SAA 250 in which the vertical distance between blood pressure cuffs may be measured using an optical sensing mechanism that includes an opposing optical emitter-detector pair (e.g., an optical emitter 274 and an optical detector 278). The SAA 250 may be used with blood pressure cuffs or other suitable blood pressure measuring devices positionable on extremities of a patient in a manner similar to that depicted, for example, in FIG. 1A. In this embodiment, a slide ring 270 may be slidably coupled to a stand rod 252. The slide ring 270 may be temporarily fixed to the stand rod 252 by a tightening knob 274. Additionally, a window strip 254 may be secured to at least a portion of the stand rod 252. The window strip 254 may include a plurality of optical windows 258 that are spaced along the window strip 254 over a predetermined range on the stand rod 252. Furthermore, the slide ring 274 may be configured such that it may slide up and down relative to the stand rod 252 and the window strip 254.

As shown in FIG. 2D, the slide ring 270 may include the optical emitter 274 and the optical detector 278 that are positioned opposite each other, with the window strip 254 positioned therebetween. In operation, the operator may first position the slide ring 270 at a reference point on the stand rod 252. The reference may, for example, be the top of the stand rod 252, the bottom of the stand rod 252, or a point in between such as a designated one of the optical windows 258. Then, as the operator adjusts the slide ring 270 relative to the stand rod 252 to fit an individual patient, the optical emitter-detector pair 274, 278 may operate to sense the position of the slide ring 270. For example, the emitter-detector pair 274, 278 may track the number of optical windows 258 traversed from the reference point when the operator adjusts the position of the slide ring 270 based on, for example, reception of light pulses by the detector 278 through the optical windows 258 as the emitter 274 transmits a light signal while the position of the slide ring 270 is adjusted. Alternatively or additionally, the optical windows 258 may include properties (e.g., shape, size, spacing, or the like) that enable the SAA to determine the position of the slide ring 270 on the stand rod 252 using information received from the optical emitter-detector pair 274, 278. Additionally, the optical emitter-detector pair 274, 278 may communicate this information to a computational device (not shown) via a cable 262. The computational device may be operable to output the height of a blood pressure cuff or the vertical distance between two blood pressure cuffs. Furthermore, the computational device may be operable to determine the HCF and/or the ABI for the patient.

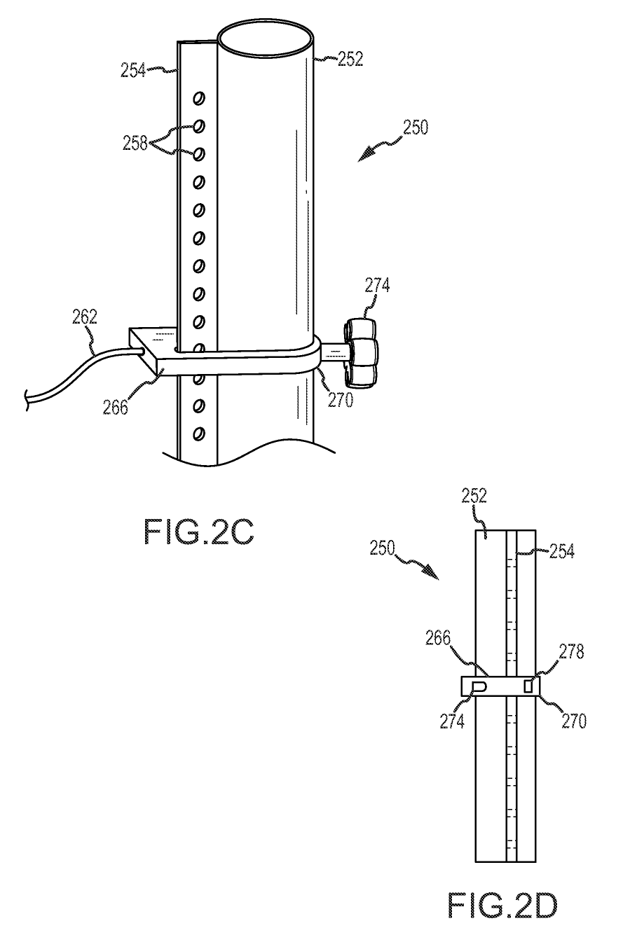

FIG. 3 illustrates an embodiment of an SAA 300 that uses acoustic signals to measure the vertical distance between two blood pressure cuffs. The SAA 300 may be used with blood pressure cuffs or other suitable blood pressure measuring devices positionable on extremities of a patient in a manner similar to that depicted, for example, in FIG. 1A. Similar to previously described embodiments, a slide ring 312 may be slidably coupled to a stand rod 304. The height of the slide ring 312 may be adjusted by height adjustment knob 308. Additionally, an ultrasonic transducer 320 may be coupled to the slide ring 312. The ultrasonic transducer 320 is operable to transmit and receive acoustic signals. In operation the ultrasonic transducer 320 may transmit an acoustic signal 328 at time T1 toward a reference reflector 340 that is at a known height. The transmitted acoustic signal 328 may reflect off the reflector 340 and a return acoustic signal 332 will strike the ultrasonic transducer 320 at time T2. The ultrasonic transducer 320 may communicate the times T1 and T2 to a computational device via cable 316. The computational device may then compute the vertical height of the slide ring 312 using, for example, T1, T2, and the speed of sound in air. Once the computational device has computed the vertical distance between two blood pressure cuffs, it may determine and output the HCF and or ABI of the patient. Although the reflector 340 and ultrasonic transducer 320 are shown to be positioned on stand rod 304, it should be noted that their respective positions could be any location that may be desirable. For example, the ultrasonic transducer 320 may be fixed on the floor or ceiling of an exam room while the reflector 340 is positioned on the stand rod 304. Additionally, the reflector 340 may any physical reference object such as a wall, floor, ceiling, or the like.

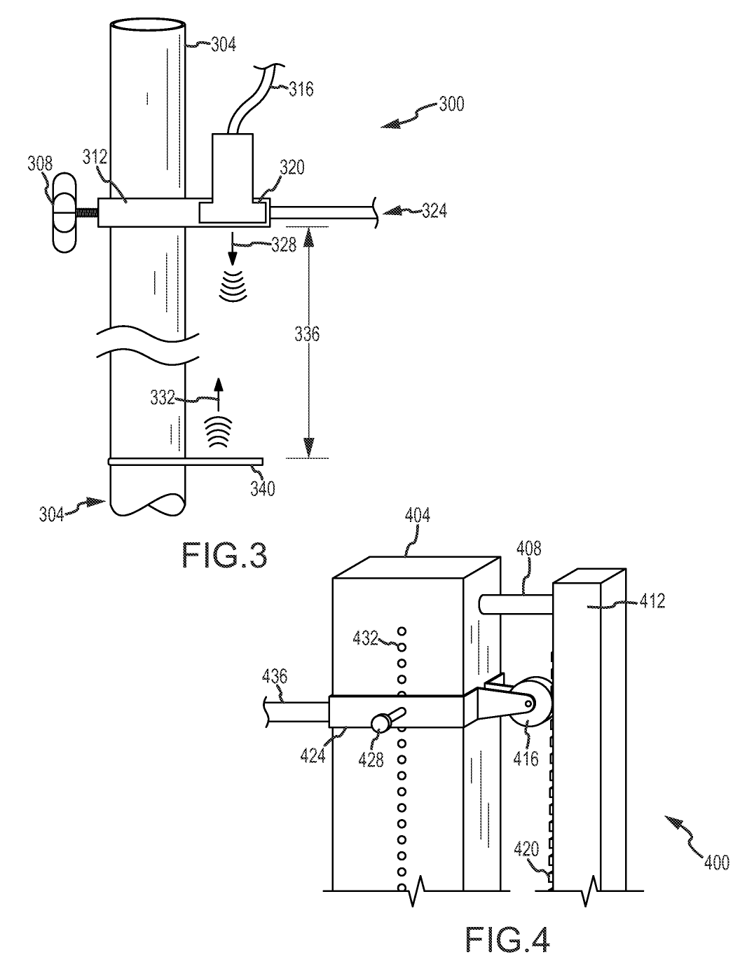

FIG. 4 illustrates an embodiment of an SAA 400 in which the SAA 400 determines the vertical distance between two blood pressure cuffs electromechanically. The SAA 400 may be used with blood pressure cuffs or other suitable blood pressure measuring devices positionable on extremities of a patient in a manner similar to that depicted, for example, in FIG. 1A. Similar to previous embodiments, a blood pressure cuff (not shown) may be coupled to a cuff-locating arm 436 which is in turn coupled to a sliding collar 424. The sliding collar 424 may be slidably disposed around a cuff stand 404. Furthermore, a pin 428 may be used to retain the position of the sliding collar 424 relative to the cuff stand 404 by inserting the pin 428 in a locating hole 432 on the cuff stand 404. A push button array switch panel 412 may be disposed in proximity to the cuff stand 404. The push button array switch panel 412 may include a plurality of push button switches 420 positioned vertically on a side of the panel 412. Furthermore, an actuator wheel 416 may be coupled to the sliding collar 424 and configured to press the push button switches 420 as the sliding collar 424 is adjusted up and down the cuff stand 404. The push button switches 420 may be electrically coupled to a circuit capable of communicating the state of each switch to a computational device. Then, the computational device may use this information to determine the height of the sliding collar 424 and the blood pressure cuff.

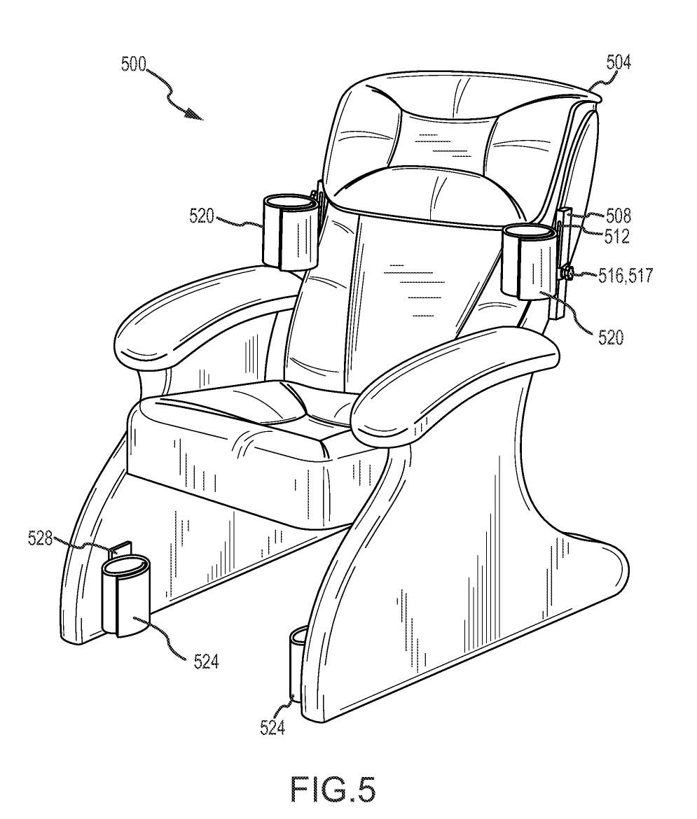

FIG. 5 illustrates an embodiment of an SAA 500 in which the vertical distance measuring system is built into a patient exam chair 504. In this embodiment, ankle blood pressure measuring cuffs 524 may be mounted to the exam chair 504 by cuff mounting brackets 528. The cuff mounting brackets 528 may be fixed or movable relative to the exam chair 504. Additionally, the exam chair 504 may include arm cuff support rods 508 for supporting arm blood pressure measuring cuffs 520. The arm cuffs 520 may be slidably coupled to the arm cuff support rods 508 by a sliding bracket 512, slide ring 516, and a tightening knob 517. The vertical distance between the arm cuffs 520 and the ankle cuffs 524 may be measured using any one of the methods described herein. For example, the arm cuff support rod may be equipped with markings that permit the operator to read the vertical distance, or the SAA 500 may employ optical or acoustic sensing devices to measure and communicate the vertical distance between the cuffs 520, 524.

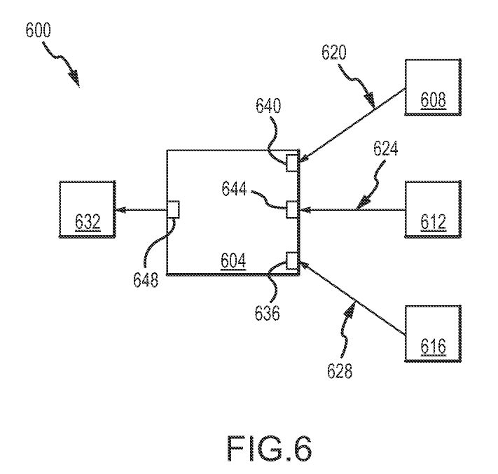

FIG. 6 illustrates an embodiment of a system 600 that may be operable to determine the hydrostatic correction factor and/or the ABI or other EBPR of a patient. The system 600 includes a computational device 604 (e.g., a processor and related components such as, for example, a memory storing instructions executable by the processor) having an input 636 capable of receiving at least information relating to a vertical distance between two blood pressure measuring devices (e.g., blood pressure cuffs 608, 612). The vertical distance information may be received by the computational device 604 from a vertical distance measuring device 616. The vertical distance measuring device 616 may be any device that provides the vertical distance information. For example, the vertical distance measuring device 616 may be the SAA 100 described above. Accordingly, the operator may read the vertical distance information from the SAA 100 and input the vertical distance information to the computational device 604 using a suitable input device (connectable to the computational device 604 via input 636 for example) such as, for example, a keyboard, a keypad, a mouse, a touch-screen, a microphone (e.g., with voice recognition software executed by the computational device 604 or an interface device) or the like. The operator may also physically measure or estimate the vertical distance without the assistance of an SAA (e.g., using a tape measure or the like) and input the distance information using a suitable input device (connectable to the computational device 604 via input 636 for example) such as, for example, a keyboard, a keypad, a mouse, a touch-screen, a microphone (e.g., with voice recognition software executed by the computational device 604 or an interface device) or the like. The vertical distance measuring device 616 may also be a device such as one of the embodiments described above that electrically communicates the vertical distance information to the computational device 604 via a communication link 628.

Once the computational device 604 has received the vertical distance information, it may compute the HCF using the specific gravities of blood and mercury, as described above. The computational device 604 may then output the result via an output element 632. The output element 632 may be operable to communicate the HCF to an operator visually, audibly, or in any other suitable manner. For example the output element may comprise a monitor. Alternatively or additionally, the output element 632 may comprise a speaker system that audibly notifies the operator of the HCF. In addition to determining the HCF, the system 600 may also be operable to calculate the corrected ABI for a patient. In this configuration, the computational device 604 may be operable to receive blood pressure measurements from blood pressure cuffs 608, 612. The computational device 604 may receive the blood pressure measurements and/or vertical distance information from the operator entering the measurements into the computational device 604 using a suitable input device (connectable to the computational device 604 via input 636 for example) such as, for example, a keyboard, a keypad, a mouse, a touch-screen, a microphone (e.g., with voice recognition software executed by the computational device 604 or an interface device) or the like. The blood pressure cuffs 608, 612 may also be configured to communicate with the computational device 604 via the communication links 620, 624. The communication links 620 and 624 may be any suitable means for communicating blood pressure measurements (e.g., cables, wireless signals, or the like). Once the computational device 604 has received the vertical distance information between the two cuffs and the blood pressure measurements, it may then calculate the corrected ABI for the patient using the formula described above. The computational device 604 may then output the HCF and/or ABI for the patient on the output element 632. Additionally, the computational device 604 may output the vertical distance information and/or a corrected ankle pressure on the output element 632.

Although a number of the embodiments described herein are well suited for a patient in a seated position and figures depicting such embodiments show a patient in an upright seated position, each of the various embodiments may more generally be used or adapted for a patient in any non-supine position (e.g. seated in an upright position, seated in a reclined position, laying on an inclined bed, or standing).

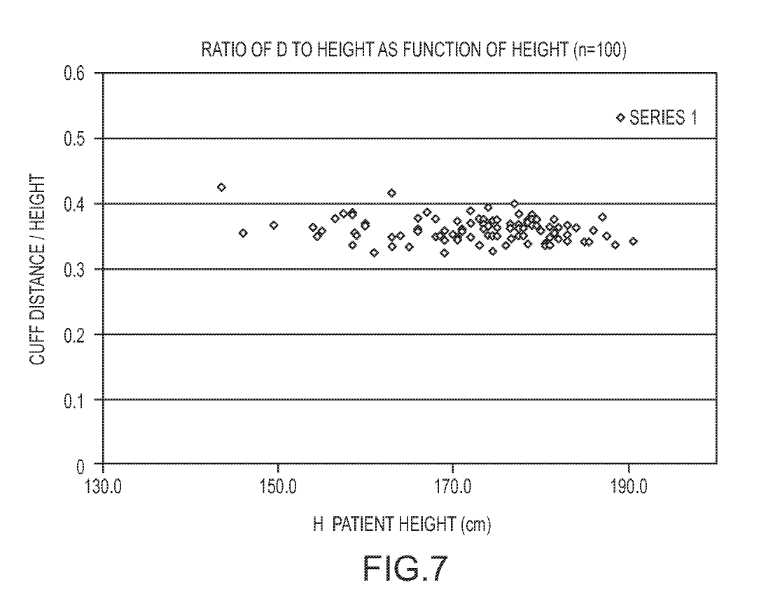

FIG. 7 is a chart that illustrates empirical data of the ratio of the vertical distance between blood pressure cuffs when a patient is seated to their height. From the chart, it is evident that for a majority of patients, the vertical distance will be between 32 percent and 40 percent of their height. Therefore, in some applications where an estimated vertical distance is sufficient, a computational device or an operator may calculate the HCF and/or ABI for a seated patient using a predetermined HCF by using an empirically derived formula for the vertical distance using a percentage within such a range (e.g., vertical distance=patient height×0.36). In this embodiment, the computational device or the operator may receive, for example, ankle and arm blood pressure measurements for a patient in a non-supine position and determine the ABI using the predetermined HCF.

The data depicted in FIG. 7 was derived for a patient seated in a generally upright position (e.g., in a non-reclined chair). The typical range of percentages of the vertical distance between blood pressure cuffs to the patient's height may vary from the percentage range derived from the data shown in FIG. 7 when the patient is not seated in a generally upright position. For example, where the patient is in a more reclined position (e.g., seated in a reclined chair or laying on inclined bed), the empirically derived formula that is used to estimate the vertical distance between blood pressure cuffs may, for example, be based on a value in the range of 8 percent to 14 percent of the patient's height (e.g., vertical distance=patient height×0.12). By way of further example, where the patient standing, the empirically derived formula that is used to estimate the vertical distance between blood pressure cuffs may, for example, be based on a value in the range of 57 percent to 63 percent of the patient's height (e.g., vertical distance=patient height×0.60).

Regardless of whether the patient is seated in an upright position, seated in a reclined position, laying on an inclined bed, standing or is otherwise in a non-supine position, the empirically derived formula may employ appropriate percentages outside of the exemplary ranges mentioned previously or any specific percentage within an appropriate range. Furthermore, the empirically derived formula may employ a measurable characteristic of the patient other than their height, such as for example, the distance from their left fingertips to their right fingertips when their arms are raised and outstretched in opposite directions from their side.

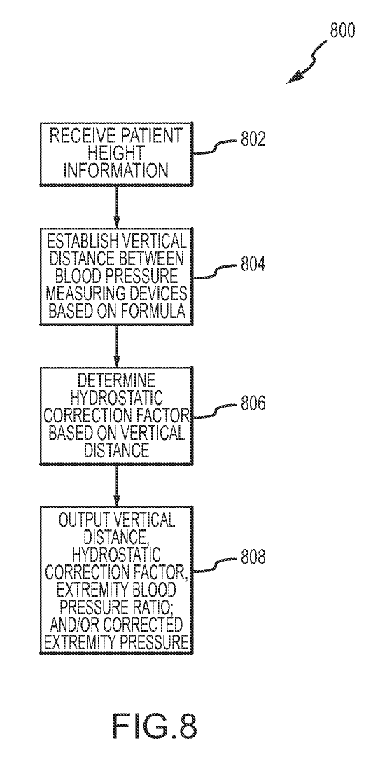

FIG. 8 depicts the steps involved in one embodiment of a method (800) for facilitating the determination of a hydrostatic correction factor for an extremity blood pressure ratio examination of a patient in a non-supine position. In this regard, a first blood pressure measuring device may be positioned to measure a blood pressure present in a first extremity of the patient and a second blood pressure measuring device may be positioned to measure a blood pressure present in a second extremity of the patient, with one of the blood pressure measuring devices being located above the other blood pressure measuring device. The method (800) includes the step (802) wherein information about a height of the patient is received. The height information may be received in a number of manners including, for example, from a clinician that measures the patient's height. In step (804) the vertical distance between the first and second blood pressure measuring devices is established based on an empirically derived formula such as, for example, a formula based on a percentage of the height of the patient. In step (806), a hydrostatic correction factor for the extremity blood pressure ratio examination is determined based on the vertical distance. In this regard, the hydrostatic correction factor may, for example, be obtained by multiplying the vertical distance by the ratio of the specific gravities of blood and mercury. In step (808) one or more of the vertical distance, the hydrostatic correction factor, an EBPR, and a corrected extremity pressure of the patient may be output. In this regard, the hydrostatic correction factor can be subtracted from the measured extremity blood pressure to provide a corrected extremity blood pressure. Subsequently, the EBPR for a patient may be calculated using conventional methods. Though not a requirement, one or more of the steps of method (800) may be implemented using a computational device or the like such as, for example, microprocessor executing appropriate computer program instructions.

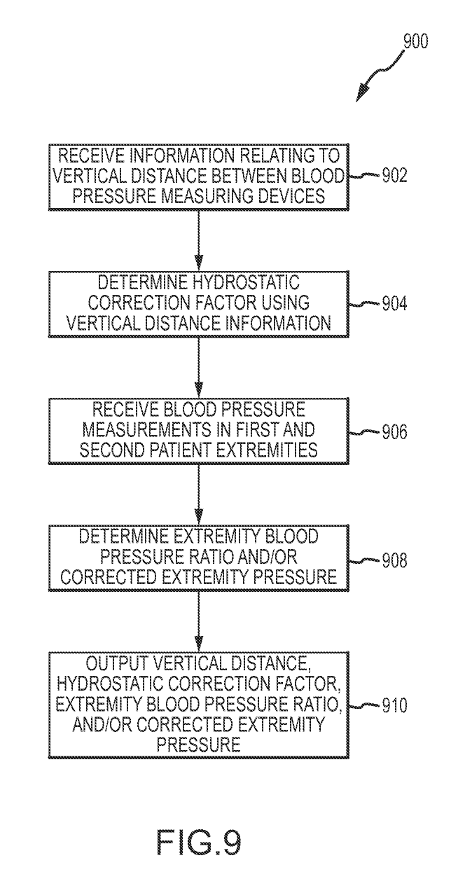

FIG. 9 depicts the steps involved in another embodiment of a method (900) for facilitating the determination of a hydrostatic correction factor for an extremity blood pressure ratio examination of a patient in a non-supine position. In this regard, a first blood pressure measuring device may be positioned to measure a blood pressure present in a first extremity of the patient and a second blood pressure measuring device may be positioned to measure a blood pressure present in a second extremity of the patient, with one of the blood pressure measuring devices being located above the other blood pressure measuring device. The method (900) includes step (902) wherein information relating to the vertical distance between the first and second blood pressure measuring devices is received. The vertical distance information may be received in a number of manners including, for example, as an input from a clinician or other individual that measures the distance and/or from a mechanism or other device that measures the distance. In step (904), a hydrostatic correction factor for the extremity blood pressure ratio examination is determined based on the vertical distance. In this regard, the hydrostatic correction factor may, for example, be obtained by multiplying the vertical distance by the ratio of the specific gravities of blood and mercury. In step (906), blood pressure measurements in the patient's first and second extremities are received from the first and second blood pressure measuring devices. In step (908), an EBPR and/or a corrected extremity pressure are determined using the blood pressure measurements and the hydrostatic correction factor. In this regard, the hydrostatic correction factor can be subtracted from the measured extremity blood pressure to provide a corrected extremity blood pressure. Subsequently, the EBPR for a patient may be calculated using conventional methods. In step (910), one or more of the vertical distance, the hydrostatic correction factor, the EBPR, and the corrected extremity pressure of the patient may be output. Though not a requirement, one or more of the steps of method (900) may be implemented using a computational device or the like such as, for example, microprocessor executing appropriate computer program instructions.

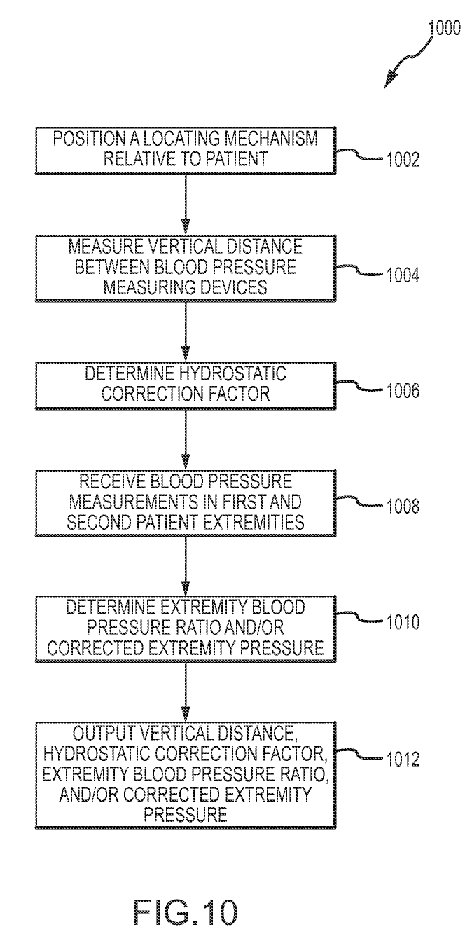

FIG. 10 depicts the steps involved in a further embodiment of a method (1000) for facilitating the determination of a hydrostatic correction factor for an extremity blood pressure ratio examination of a patient in a non-supine position. In this regard, a first blood pressure measuring device may be positioned to measure a blood pressure present in a first extremity of the patient and a second blood pressure measuring device may be positioned to measure a blood pressure present in a second extremity of the patient, with one of the blood pressure measuring devices being located above the other blood pressure measuring device. The method (1000) includes step (1002) wherein a locating mechanism is positioned relative to the patient. In this regard, the locating mechanism fixes a portion of at least one of the first and second blood pressure measuring devices positioned relative to its respective patient extremity. In step (1004), the vertical distance between the first and second blood pressure measuring devices is measured using the locating mechanism. In step (1006), a hydrostatic correction factor for the extremity blood pressure ratio examination is determined based on the vertical distance. In this regard, the hydrostatic correction factor may, for example, be obtained by multiplying the vertical distance by the ratio of the specific gravities of blood and mercury. In step (1008), blood pressure measurements in the patient's first and second extremities are received from the first and second blood pressure measuring devices. In step (1010), an EBPR and/or a corrected extremity pressure are determined using the blood pressure measurements and the hydrostatic correction factor. In this regard, the hydrostatic correction factor can be subtracted from the measured extremity blood pressure to provide a corrected extremity blood pressure. Subsequently, the EBPR for a patient may be calculated using conventional methods. In step (1012), one or more of the vertical distance, the hydrostatic correction factor, the EBPR, and the corrected extremity pressure of the patient may be output. Though not a requirement, one or more of the steps of method (1000) may be implemented using a computational device or the like such as, for example, microprocessor executing appropriate computer program instructions.

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that all changes and modifications to the described embodiments that come within the spirit of the invention are desired to be protected.

What is claimed is:

1. A method for facilitating the determination of a hydrostatic correction factor for an extremity blood pressure ratio examination of a patient in a non-supine position, wherein a first blood pressure measuring device is positionable to measure a blood pressure present in a first extremity of the patient and a second blood pressure measuring device is positionable to measure a blood pressure present in a second extremity of the patient, one of the blood pressure measuring devices being located above the other blood pressure measuring device, the method comprising the steps of:
establishing the vertical distance between the first and second blood pressure measuring devices based on an empirically derived formula; and
determining a hydrostatic correction factor for the extremity blood pressure ratio examination based on the vertical distance.

2. The method of claim 1, wherein the formula for the vertical distance is based on a percentage of the height of the patient.

3. The method of claim 2, wherein the percentage is between about 32 percent and 40 percent.

4. The method of claim 3, wherein the percentage is about 36 percent.

5. The method of claim 2, wherein the percentage is between about 8 percent and 14 percent.

6. The method of claim 5, wherein the percentage is about 12 percent.

7. The method of claim 2, wherein the percentage is between about 57 percent and 63 percent.

8. The method of claim 7, wherein the percentage is about 60 percent.

9. The method of claim 1, wherein the first extremity is an upper extremity of the patient and the second extremity is a lower extremity of the patient.

10. The method of claim 9, wherein the first blood pressure measuring device is positioned to measure blood pressure at the patient's upper arm, and the second blood pressure measuring device is positioned to measure blood pressure at the patient's ankle.

11. The method of claim 2, wherein said step of establishing comprises:
providing information about the patient's height to a computational device operable to calculate the vertical distance based on the empirically derived formula; and
operating the computational device to calculate the vertical distance.

12. The method of claim 11 wherein said step of providing comprises:
measuring the patient's height; and
inputting the patient's height to the computational device.

13. The method of claim 11 wherein said step of determining comprises:
operating the computational device to determine the hydrostatic correction factor based on the vertical distance calculated by the computational device.

14. The method of claim 13, further comprising the steps of:
providing blood pressure information from the first and second blood pressure measuring devices to the computational device;
operating the computational device to determine an extremity blood pressure ratio for the patient based on the hydrostatic correction factor.

15. The method of claim 14, wherein said step of providing blood pressure information occurs without contemporaneous human interaction.

16. The method of claim 14, further comprising the step of:
outputting from the computational device at least one of the vertical distance, the hydrostatic correction factor, the extremity blood pressure ratio, and a corrected extremity pressure of the patient.

17. A method for facilitating the determination of a hydrostatic correction factor for an extremity blood pressure ratio examination of a patient in a non-supine position, wherein a first blood pressure measuring device is positionable to measure a blood pressure present in a first extremity of the patient and a second blood pressure measuring device is positionable to measure a blood pressure present in a second extremity of the patient, one of the blood pressure measuring devices being located above the other blood pressure measuring device, the method comprising the steps of:
receiving information about a height of the patient;
establishing the vertical distance between the first and second blood pressure measuring devices based on a percentage of the height of the patient; and
determining a hydrostatic correction factor for the extremity blood pressure ratio examination based on the vertical distance.

18. The method of claim 17, wherein the percentage is between about 32 percent and 40 percent.

19. The method of claim 18, wherein the percentage is about 36 percent.

20. The method of claim 17, wherein the percentage is between about 8 percent and 14 percent.

21. The method of claim 20, wherein the percentage is about 12 percent.

22. The method of claim 17, wherein the percentage is between about 57 percent and 63 percent.

23. The method of claim 22, wherein the percentage is about 60 percent.

24. The method of claim 17, wherein the first extremity is an upper extremity of the patient and the second extremity is a lower extremity of the patient.

25. The method of claim 24, wherein the first blood pressure measuring device is positioned to measure blood pressure at the patient's upper arm, and the second blood pressure measuring device is positioned to measure blood pressure at the patient's ankle.

26. A method for facilitating the determination of a hydrostatic correction factor for an extremity blood pressure ratio examination of a patient in a non-supine position, wherein a first blood pressure measuring device is positionable to measure a blood pressure present in a first extremity of the patient and a second blood pressure measuring device is positionable to measure a blood pressure present in a second extremity of the patient, one of the blood pressure measuring devices being located above the other blood pressure measuring device, the method comprising the steps of:
receiving an input of information about a height of the patient at a computational device operable to calculate the vertical distance based on a percentage of the height of the patient; and
operating the computational device to calculate the vertical distance; and
operating the computational device to determine the hydrostatic correction factor based on the vertical distance calculated by the computational device.

27. The method of claim 26, further comprising the steps of:
receiving an input of blood pressure information from the first and second blood pressure measuring devices at the computational device;
operating the computational device to determine an extremity blood pressure ratio for the patient based on the hydrostatic correction factor.

28. The method of claim 27, wherein said step of receiving an input of blood pressure information occurs without contemporaneous human interaction.

29. The method of claim 27, further comprising the step of:
outputting from the computational device at least one of the vertical distance, the hydrostatic correction factor, the extremity blood pressure ratio, and a corrected extremity pressure of the patient.
